(19) United States
(12) Patent Application Publication
(10) Pub. No.: US 2012/0157948 A1
(43) Pub. Date: Jun. 21, 2012

(54) Absorbent Article With Indicator

(76) Inventors: David-Dang Hoang Nhan, Appleton, WI (US); William Pitman Laratta, Barrio Pinares (CR); Thomas Michael Ales, III, Neenah, WI (US); Sandra Kay Knight, Omro, WI (US)

(21) Appl. No.: 12/968,440

(22) Filed: Dec. 15, 2010

(51) Int. Cl. A61F 13/42; B32B 37/18

(52) U.S. Cl. 604/361; 29/428

(57) ABSTRACT

A disposable absorbent article and a method for providing a disposable absorbent article having a urine insult volume indicator and a longitudinal centerline having a urine insult volume indicator and a longitudinal centerline includes a liquid permeable inner surface for facing a wearer; an outer cover having an outer surface for facing away from the wearer; an absorbent body disposed therebetween; and a front waist region, a back waist region, and a crotch region extending longitudinally between and interconnecting the front and back waist regions. The article also includes a two-dimensional pattern having a plurality of segments, a longitudinal extent, and a transverse extent, the pattern disposed between the absorbent core and the outer surface, wherein the pattern is in fluid contact with the absorbent core, and wherein a segment changes appearance when wetted and is visible from the outer surface when wetted.

### BACKGROUND

Various absorbent articles, such as disposable diapers, are commercially available with wetness indicators. The indicators identify when a diaper has received a substantial quantity of liquid and should be changed. For example, these indicators can include the appearance or disappearance of a design on the outer surface of the diaper that is visually identifiable at a distance. Other absorbent articles include indicators that identify the presence of various substances in the urine, such as protein.

However, newborn babies generally have very small urine discharges of about 10 to about 15 cubic centimeters (cc). Due to the advanced technology of disposable diapers and their ability to keep the baby dry and clean, it can be difficult to detect whether any insult has occurred. As a result, it can be difficult for parents to monitor the child's quantity and frequency of urination. This information can be helpful in diagnosing whether an infant/child is dehydrated (a common cause of newborn babies being readmitted to the hospital after birth).

Dehydration is a significant concern when caring for premature and other babies while being treated in neonatal intensive care. Typically healthcare workers need to use a number of methods to monitor hydration status of such babies, and often more than one technique is used. Methods for evaluating hydration status of infants in intensive care include weighing diapers to determine urine volume, or measuring urine parameters such as osmolarity, specific gravity, anion gap, or urea concentration. Each of these techniques has particular drawbacks. For instance, techniques such as osmolarity, specific gravity, anion gap, and urea concentration require collection of urine samples, which is difficult to achieve in patients that cannot be asked to provide a sample upon demand. Thus, obtaining urine samples from these patients often requires catheterization, attachment of collection bags, or similarly invasive techniques. The most commonly cited method for determining hydration status of babies receiving neonatal intensive care involves weighing diapers at timed intervals to determine urine volume. Babies producing a sufficient volume of urine over time are presumed to be adequately hydrated. This diaper weighing technique also has a number of drawbacks, including the need to disturb the baby each time the diaper is changed and the additional workload demands placed on the neonatal healthcare providers.

Skin health of babies or toddlers is also a concern for caregivers. A soiled diaper or pants should not be in contact with a wearer for an extended length of time. Thus, a diaper or training pants with a “time to change” indicator would provide a higher order of benefit to caregivers. This type of indicator would be very useful to tell caregivers when a diaper or training pants should be changed to keep their kid's skin healthy. Furthermore, the indicator can help to minimize unnecessary diaper change; thus it provides a value benefit to consumers, especially in lower income markets.

To address the dehydration and skin health concerns stated above, some diapers and training pants in the market have a wetness indicator to signal caregivers that urination occurs. A variety of different methods are used to construct a wetness indicator on a product. These methods can range from fading graphics, to color changing graphics, to appearing graphics. A main problem with the existing wetness indicator in current diapers and training pants is that they can leach ink onto the skin of a user, especially when a wetness indicator is designed to be viewed from the inside of a pants/diapers. Furthermore, the current diapers/pants with wetness indicator do not provide urine insult volume information to caregivers.

### SUMMARY

Hence, there is a need to be able to identify whether or not small insults have occurred and to determine whether various substances exist in urine, a need for solutions that do not require removal and weighing of the diaper. The fullness indicator or absorbent saturation level indicator of the present disclosure solves these shortcomings by using a correlation between wetted length or area and urine insult volume. The present disclosure describes an absorbent article having an easy-to-interpret visual indicator of urine volume. In this case, urine volume within an absorbent article correlates to a visual signal seen outside of the absorbent article.

To accomplish this, the present disclosure uses wetness-responsive-inks that can be printed onto the outer cover material or other structure of the article. When urine enters the article and contacts the ink(s), the ink(s) respond either by changing color, appearing, or disappearing. The appearance change is visible outside of the article, thus providing a visual signal. These ink(s) are printed on the article in a specified pattern that permits a healthcare professional or caregiver to immediately see or to make a correlation between the location of an appearance change and the volume of urine in the article. In a non-limiting example, the ink(s) can be printed in a series of concentric oval-shaped objects, where the size and position of each oval corresponds to a different volume of urine. In other non-limiting aspects, the ink(s) can be printed in specific grid patterns or in random patterns that cover the diaper/training pants surface. Urine volume in the diaper/training pants can be estimated by visually observing an appearance change. Healthcare professionals or caregivers are able to estimate the volume of urine in such articles by simply observing where the change occurs on the outer cover.

Disclosed herein are absorbent articles and absorbent article systems. In one aspect, a disposable absorbent article having a urine insult volume indicator and a longitudinal centerline includes a liquid permeable inner surface for facing a wearer; an outer cover having an outer surface for facing away from the wearer; an absorbent body disposed therebetween; and a front waist region, a back waist region, and a crotch region extending longitudinally between and interconnecting the front and back waist regions. The article also includes a two-dimensional pattern having a plurality of segments, a longitudinal extent, and a transverse extent, the pattern disposed between the absorbent core and the outer surface, wherein the pattern is in fluid contact with the absorbent core, and wherein a segment changes appearance when wetted and is visible from the outer surface when wetted.

In another aspect, a method for providing a disposable absorbent article having a urine insult volume indicator and a longitudinal centerline includes manufacturing a disposable absorbent article including a liquid permeable inner surface for facing a wearer; an outer cover having an outer surface for facing away from the wearer; an absorbent body disposed therebetween; and a two-dimensional pattern having a plurality of segments, a longitudinal extent, and a transverse extent, the pattern disposed between the absorbent core and the outer surface, wherein the pattern is in fluid contact with the absorbent core, and wherein a segment changes appearance when wetted and is visible from the outer surface when wetted; and correlating the number of segments that change appearance upon wetting to a urine insult volume resident in the absorbent core.

Other features of the disclosure will be in part apparent and in part pointed out hereinafter. Other objects and advantages of the present disclosure will become more apparent to those skilled in the art in view of the following description and the accompanying drawings.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 representatively shows a partially cut away, top plan view of an absorbent article in a stretched and laid flat condition with the surface of the article that contacts the skin of the wearer facing the viewer;

FIG. 2 representatively shows a sectional view of the absorbent article of FIG. 1 taken along line 2-2;

FIG. 3 representatively illustrates a urine insult indicator pattern on the absorbent article of FIG. 1;

FIG. 4 representatively illustrates an alternative urine insult indicator pattern on the absorbent article of FIG. 1;

FIG. 5 representatively illustrates another alternative urine insult indicator pattern on the absorbent article of FIG. 1;

FIG. 6 representatively illustrates another alternative urine insult indicator pattern on the absorbent article of FIG. 1;

FIG. 7 representatively illustrates another alternative urine insult indicator pattern on the absorbent article of FIG. 1;

FIG. 8 representatively illustrates another alternative urine insult indicator pattern on the absorbent article of FIG. 1;

FIG. 9 representatively illustrates another alternative urine insult indicator pattern on the absorbent article of FIG. 1;

FIG. 10 representatively illustrates another alternative urine insult indicator pattern on the absorbent article of FIG. 1;

FIG. 11 representatively illustrates a photographic and x-ray analysis of the results of testing sample products of the type of FIG. 1;

FIG. 12 representatively illustrates a photographic analysis of the results of testing a sample product of the type of FIG. 1;

FIG. 13 is a representation of the linear correlation between wetted length and insult amount resulting from testing products of the type of FIG. 1;

FIG. 14 is a representation of the linear correlation between wetted area as obtained from x-ray analysis and wetted area as obtained from counting the number of squares that changed appearance resulting from testing products of the type of FIG. 1;

FIG. 15 is a representation of the linear correlation between wetted length as obtained from x-ray analysis and wetted length as obtained from counting the number of rows with squares that changed appearance resulting from testing products of the type of FIG. 1; and

FIG. 16 is a representation of the linear correlation between insult amount and the first and last rows with squares that changed appearance resulting from testing products of the type of FIG. 1.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

It is to be understood by one of ordinary skill in the art that the present discussion is a description of exemplary aspects of the present disclosure only, and is not intended as limiting the broader aspects of the present disclosure.

It is noted that all ranges disclosed herein are inclusive and combinable (e.g., ranges of “up to about 25 wt %, or, more specifically about 5 wt % to about 20 wt %” is inclusive of the endpoints and all intermediate values of the ranges of “about 5 wt % to about 25 wt %,” etc.). The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced items. Unless defined otherwise, technical and scientific terms used herein have the same meaning as is commonly understood by one of skill in the art to which this disclosure belongs. The modifier “about” used in connection with a quantity is inclusive of the stated value and has the meaning dictated by the context, (e.g., includes the degree of error associated with measurement of the particular quantity).

Disclosed herein are absorbent articles and systems for determining the existence of an insult, and for estimating a volume of a liquid in absorbent articles. The absorbent articles can include a colorant having a first state when the absorbent article is dry and a second state when the absorbent article is wetted with a volume of a liquid. A difference between the first state and the second state can indicate the existence of an insult and/or the volume of the liquid. Therefore, the absorbent article can include one or more colors. In general, the greater the volume of liquid present, the greater the change in state, thus providing a relative visual indication of the volume of the liquid.

In addition, or alternatively, the colorant can have a reaction with the insult or a component thereof, such that a color change occurs. The colorant can be white or essentially colorless such that only the color of the article is seen, and change to one or more colors (e.g., blue, green, yellow, etc.) upon contact with a particular liquid, such as urine. Alternatively, the color change can start with one color and change to another (e.g., from blue to green).

The colorant is desirably a color visible to the human eye, thereby enabling identification of an insult without additional equipment (e.g., ultraviolet light, or the like). Also, due to the use of colorants in an absorbent article, it is desirable that the color be a material approved by the U.S. Food and Drug Administration (FDA) for use in food. Optionally, the colors can be various colors corresponding to different color hues of the same base color. For example, the colors can be a progression from light blue to dark blue wherein the hue of the color indicates the volume of the insult, for example.

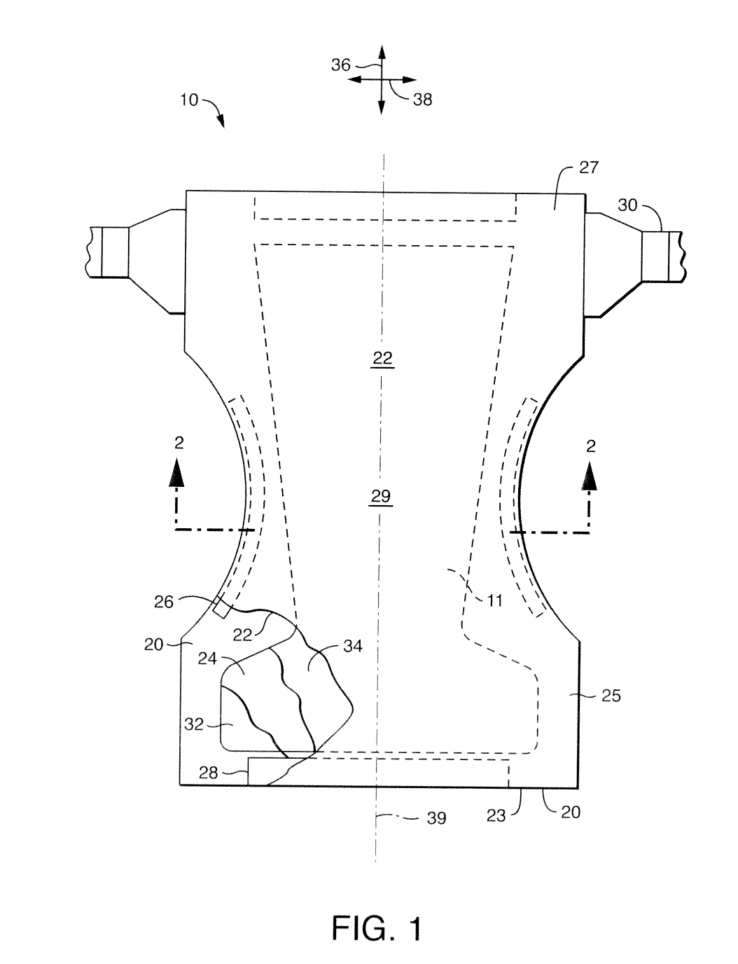

Referring now to FIG. 1, in one aspect, the absorbent article is a disposable article 10 including a backsheet or outer cover 20, a liquid permeable topsheet or bodyside liner 22 positioned in facing relation with the outer cover 20, and an absorbent body 24, such as an absorbent pad, that is located between the bodyside liner 22 and the outer cover 20. The article 10 has an outer surface 23, a front waist region 25, a back waist region 27, and a crotch region 29 connecting the front and back waist regions 25, 27. The outer cover 20 defines a length and a width that, in the illustrated aspect, coincide with the length and width of the article 10. The absorbent body 24 generally defines a length and width that are less than the length and width of the outer cover 20, respectively. Thus, marginal portions of the article 10, such as marginal sections of the outer cover 20, can extend past the terminal edges of the absorbent body 24. In the illustrated aspects, for example, the outer cover 20 extends outwardly beyond the terminal marginal edges of the absorbent body 24 to form side margins and end margins of the article 10. The bodyside liner 22 is generally coextensive with the outer cover 20 but can optionally cover an area that is larger or smaller than the area of the outer cover 20, as desired. In other words, the bodyside liner 22 is connected in superposed relation to the outer cover 20. The outer cover 20 and bodyside liner 22 are intended to face the garment and body of the wearer, respectively, while in use.

To provide improved fit and to help reduce leakage of body exudates from the article 10, the article side margins and end margins can be elasticized with suitable elastic members, such as single or multiple strands of elastic. The elastic strands can be composed of natural or synthetic rubber and can optionally be heat shrinkable or heat elasticizable. For example, as representatively illustrated in FIG. 1, the article 10 can include leg elastics 26 that are constructed to operably gather and shirr the side margins of the article 10 to provide elasticized leg bands that can closely fit around the legs of the wearer to reduce leakage and provide improved comfort and appearance. Similarly, waist elastics 28 can be employed to elasticize the end margins of the article 10 to provide elasticized waists. The waist elastics 28 are configured to operably gather and shirr the waist sections to provide a resilient comfortably close fit around the waist of the wearer. In the illustrated aspects, the elastic members are illustrated in their uncontracted, stretched condition for the purpose of clarity.

Fastening means, such as hook and loop fasteners 30, can be employed to secure the article 10 on a wearer. Alternatively, other fastening means, such as buttons, pins, snaps, adhesive tape fasteners, cohesives, mushroom-and-loop fasteners, a belt, and so forth, as well as combinations including at least one of the foregoing fasteners can be employed. Additionally, more than two fasteners can be provided, particularly if the article 10 is to be provided in a prefastened configuration.

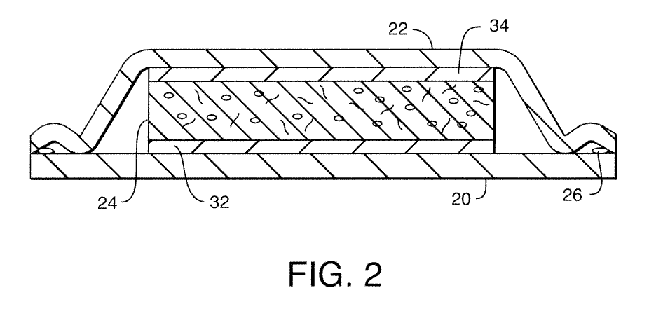

The article 10 can further include other layers between the absorbent body 24 and the bodyside liner 22 or outer cover 20. For example, as representatively illustrated in FIGS. 1 and 2, the article 10 can include a ventilation layer 32 located between the absorbent body 24 and the outer cover 20 to insulate the outer cover 20 from the absorbent body 24, to improve air circulation and to effectively reduce the dampness of the garment facing surface of the outer cover 20. The ventilation layer 32 can also assist in distributing fluid exudates to portions of the absorbent body 24 that do not directly receive the insult. The article 10 can also include a surge management layer 34 located between the bodyside liner 22 and the absorbent body 24 to prevent pooling of the fluid exudates and further improve air exchange and distribution of the fluid exudates within the article 10.

The article 10 can be of various suitable shapes. For example, the article 10 can have an overall rectangular shape, T-shape or an approximately hourglass shape. In the shown aspect, the article 10 has a generally I-shape. The article 10 further defines a longitudinal direction 36 and a transverse direction 38. Other suitable article components that can be incorporated on absorbent articles include containment flaps, waist flaps, elastomeric side panels, and the like. Examples of possible article configurations are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,192,606 issued Mar. 9, 1993, to Proxmire et al., and U.S. Pat. No. 5,509,915 issued Apr. 23, 1996 to Hanson et al.

The various components of the article 10 are integrally assembled employing various types of attachment mechanisms such as adhesive, sonic bonds, thermal bonds, and so forth, as well as combinations including at least one of foregoing mechanisms. In the shown aspect, for example, the bodyside liner 22 and outer cover 20 are assembled to the absorbent body 24 with lines of adhesive, such as a hot melt, pressure-sensitive adhesive. Similarly, other article components, such as the elastic members 26 and 28, fastening members 30, and ventilation and surge layers 32 and 34 can be assembled into the article 10 by employing the above-identified attachment mechanisms.

The outer cover 20 of the article 10 can include any material used for such applications, such as a substantially vapor-permeable material. The permeability of the outer cover 20 can be configured to enhance the breathability of the article 10 and to reduce the hydration of the wearer's skin during use without allowing excessive condensation of vapor, such as urine, on the garment facing surface of the outer cover 20 that can undesirably dampen the wearer's clothes. The outer cover 20 can be constructed to be permeable to at least water vapor and can have a water vapor transmission rate of greater than or equal to about 1,000 grams per square meter per 24 hours (g/m2/24 hr). For example, the outer cover 20 can define a water vapor transmission rate of about 1,000 to about 6,000 g/m2/24 hr.

The outer cover 20 is also desirably substantially liquid impermeable. For example, the outer cover 20 can be constructed to provide a hydrohead value of greater than or equal to about 60 centimeters (cm), or, more specifically, greater than or equal to about 80 cm, and even more specifically, greater than or equal to about 100 cm. A suitable technique for determining the resistance of a material to liquid penetration is Federal Test Method Standard (FTMS) 191 Method 5514, dated Dec. 31, 1968.

As stated above, the outer cover 20 can include any material used for such applications, and desirably includes materials that either directly provide the above desired levels of liquid impermeability and air permeability and/or materials that can be modified or treated in some manner to provide such levels. The outer cover 20 can be a nonwoven fibrous web constructed to provide the required level of liquid impermeability. For example, a nonwoven web including spunbond and/or meltblown polymer fibers can be selectively treated with a water repellent coating and/or laminated with a liquid impermeable, vapor permeable polymer film to provide the outer cover 20. In another aspect, the outer cover 20 can include a nonwoven web including a plurality of randomly deposited hydrophobic thermoplastic meltblown fibers that are sufficiently bonded or otherwise connected to one another to provide a substantially vapor permeable and substantially liquid impermeable web. The outer cover 20 can also include a vapor permeable nonwoven layer that has been partially coated or otherwise configured to provide liquid impermeability in selected areas. In yet another example, the outer cover 20 is provided by an extensible material. Further, the outer cover 20 material can have stretch in the longitudinal 36 and/or lateral 38 directions. When the outer cover 20 is made from extensible or stretchable materials, the article 10 provides additional benefits to the wearer including improved fit.

The bodyside liner 22, employed to help isolate the wearer's skin from liquids held in the absorbent body 24, can define a compliant, soft, non-irritating feel to the wearer's skin. Further, the bodyside liner 22 can be less hydrophilic than the absorbent body 24, to present a relatively dry surface to the wearer, and can be sufficiently porous to be liquid permeable, permitting liquid to readily penetrate through its thickness. A suitable bodyside liner 22 can be manufactured from a wide selection of web materials, such as porous foams, reticulated foams, apertured plastic films, natural fibers (for example, wood or cotton fibers), synthetic fibers (for example, polyester or polypropylene fibers), and the like, as well as a combination of materials including at least one of the foregoing materials.

Various woven and nonwoven fabrics can be used for the bodyside liner 22. For example, the bodyside liner 22 can include a meltblown or spunbond web (e.g., of polyolefin fibers), a bonded-carded web (e.g., of natural and/or synthetic fibers), a substantially hydrophobic material (e.g., treated with a surfactant or otherwise processed to impart a desired level of wettability and hydrophilicity), and the like, as well as combinations including at least one of the foregoing. For example, the bodyside liner 22 can include a nonwoven, spunbond, polypropylene fabric, optionally including about 2.8 to about 3.2 denier fibers formed into a web having a basis weight of about 22 grams per square meter (g/m²) and a density of about 0.06 gram per cubic centimeter (g/cc).

The absorbent body 24 of the article 10 can include a matrix of hydrophilic fibers, such as a fibrous web of cellulosic fibers, mixed with particles of a high-absorbency material (such as the material commonly known as superabsorbent material). The wood pulp fluff can be exchanged with synthetic, polymeric, meltblown fibers, and the like, as well as a combination including at least one of the foregoing. The superabsorbent particles can be substantially homogeneously mixed with the hydrophilic fibers or can be nonuniformly mixed. Alternatively, the absorbent body 24 can include a laminate of fibrous webs and superabsorbent material and/or a suitable matrix for maintaining the superabsorbent material in a localized area.

The absorbent body 24 can include a fibrous web (e.g., including cellulosic fibers) and a superabsorbent material. When the absorbent body 24 includes a combination of hydrophilic fibers and high-absorbency particles, the hydrophilic fibers and high-absorbency particles can form an average basis weight for the absorbent body 24 that can be about 400 grams per square meter (g/m²) to about 900 g/m², or, more specifically, about 500 g/m² to about 800 g/m², and even more specifically, about 550 g/m² to about 750 g/m².

The high-absorbency material (e.g., superabsorbent) can be natural, synthetic, and modified natural polymers and materials; inorganic materials (such as silica gels); organic compounds (such as crosslinked polymers); and the like, as well as combinations including at least one of the foregoing. The term “crosslinked” refers to methods for effectively rendering normally water-soluble materials substantially water insoluble but swellable. Such methods include, but are not limited to, physical entanglement, crystalline domains, covalent bonds, ionic complexes and associations, hydrophilic associations such as hydrogen bonding, and/or hydrophobic associations or Van der Waals forces. Examples of high-absorbency materials include, but are not limited to, the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and the like, as well as copolymers and combinations including at least one of the foregoing. Further polymers suitable for use in the absorbent body 24 include, but are not limited to, polymers (natural and modified natural), such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and so forth. Mixtures of natural and wholly or partially synthetic absorbent polymers can also be useful. Similarly useful are various copolymers and, combinations including at least one of any of the above high-absorbency materials. An example of high-absorbency material is DRYTECH 2035 polymer available from Dow Chemical, a business having offices in Midland, Mich. Other suitable superabsorbents can include FAVOR SXM 880 polymer obtained from Stockhausen, a business having offices in Greensboro, N.C.

The high absorbency material can be in any of a wide variety of geometric forms. Generally, it is preferred that the high absorbency material be in the form of discrete particles. However, the high absorbency material can also be in the form of fibers, flakes, rods, spheres, needles, particles, or the like, as well as combinations including at least one of the foregoing. In general, the high absorbency material is present in the absorbent body 24 in an amount of greater than or equal to about 5 weight percent (wt %), or, more specifically greater than or equal to about 30 wt %, and even more specifically, greater than or equal to about 50 wt % based on a total weight of the absorbent body 24. For example, in a particular aspect, the absorbent body 24 can include a laminate that includes greater than or equal to about 50 wt %, or, more specifically, greater than or equal to about 70 wt % of high-absorbency material overwrapped by a fibrous web or other suitable material for maintaining the high-absorbency material in a localized area.

Optionally, the absorbent body 24 can further include a support (e.g., a substantially hydrophilic tissue or nonwoven wrapsheet (not illustrated)) to help maintain the integrity of the structure of the absorbent body 24. The tissue wrapsheet can be placed about the web/sheet of high-absorbency material and/or fibers, optionally over at least one or both major facing surfaces thereof. The tissue wrapsheet can include an absorbent cellulosic material, such as creped wadding or a high wet-strength tissue. The tissue wrapsheet can optionally be configured to provide a wicking layer that helps to rapidly distribute liquid over the mass of absorbent fibers constituting the absorbent body 24. If this support is employed, the colorant 40 can optionally be disposed in the support, on the side of the absorbent body 24 opposite the outer cover 20.

Due to the thinness of absorbent body 24 and the high absorbency material within the absorbent body 24, the liquid uptake rates of the absorbent body 24, by itself, can be too low, or cannot be adequately sustained over multiple insults of liquid into the absorbent body 24. To improve the overall liquid uptake and air exchange, the article 10 can further include a porous, liquid-permeable layer of surge management material 34, as representatively illustrated in FIG. 1. The surge management layer 34 is typically less hydrophilic than the absorbent body 24, and can have an operable level of density and basis weight to quickly collect and temporarily hold liquid surges, to transport the liquid from its initial entrance point and to substantially completely release the liquid to other parts of the absorbent body 24. This configuration can help prevent the liquid from pooling and collecting on the portion of the article 10 positioned against the wearer's skin, thereby reducing the feeling of wetness by the wearer. The structure of the surge management layer 34 can also enhance the air exchange within the article 10.

Various woven and nonwoven fabrics can be used to construct the surge management layer 34. For example, the surge management layer 34 can be a layer including a meltblown or spunbond web of synthetic fibers (such as polyolefin fibers); a bonded-carded-web or an airlaid web including, for example, natural and/or synthetic fibers; hydrophobic material that is optionally treated with a surfactant or otherwise processed to impart a desired level of wettability and hydrophilicity; and the like, as well as combinations including at least one of the foregoing. The bonded-carded-web can, for example, be a thermally bonded web that is bonded using low melt binder fibers, powder, and/or adhesive. The layer can optionally include a mixture of different fibers. For example, the surge management layer 34 can include a hydrophobic, nonwoven material having a basis weight of about 30 to about 120 g/m².

As representatively illustrated in FIG. 1, the article 10 can optionally also include a ventilation layer 32 located between the outer cover 20 and the absorbent body 24. The ventilation layer 32 can serve to facilitate the movement of air within and through the article 10 and to prevent the outer cover 20 from being in surface to surface contact with at least a portion of the absorbent body 24. The ventilation layer 32 can serve as a conduit through which air and water vapor can move from the absorbent body 24 through the vapor permeable outer cover 20.

The ventilation layer 32 can be formed from materials described above as being suitable for the surge management layer 34 such as nonwoven, (e.g., spunbond, meltblown, carded, and the like), woven, knitted fibrous webs, and the like, including natural fibers, synthetic polymeric fibers, and/or the like. Suitable fibers include, for example, acrylic fibers, polyolefin fibers, polyester fibers, and the like, as well as blends including at least one of the foregoing fibers. The ventilation layer 32 can also be formed from a porous foam material such as an open-celled polyolefin foam, a reticulated polyurethane foam, and the like. The ventilation layer 32 can include a single layer of material or a composite of two or more layers of material. For example, the ventilation layer 32 can include a hydrophobic, nonwoven material having a thickness of greater than or equal to about 0.10 centimeters (cm) determined under a restraining pressure of 0.34 kiloPascals (kPa) and a basis weight of about 20 g/m² to about 120 g/m². For example, the ventilation layer 32 can include a bonded-carded-web, nonwoven fabric with bicomponent fibers, and defining an overall basis weight of about 83 g/m². The ventilation layer 32 in such a configuration can be a homogeneous blend including about 60 wt % polyethylene/polyester (PE/PET), sheath-core bicomponent fibers that have a fiber denier (d) of about 3 d, and about 40 wt % single component polyester fibers that have a fiber denier of about 6 d and fiber lengths of about 3.8 cm to about 5.08 cm.

The ventilation layer 32 can be any desired shape. Suitable shapes include for example, circular, rectangular, triangular, trapezoidal, oblong, dog-boned, hourglass-shaped, oval, and the like. The ventilation layer 32 can extend beyond, completely over, or partially over a side of the absorbent body 24 disposed opposite the bodyside liner 22. For example, the ventilation layer 32 can suitably be located over the intermediate section 16 of the article 10 and be substantially centered side-to-side with respect to the longitudinal centerline 39 of the article 10. It is generally desired that the entire absorbent body 24 be overlaid with the ventilation layer 32 to prevent substantially all surface to surface contact between the outer cover 20 and the absorbent body 24. In the illustrated figures, the ventilation layer 32 is coextensive with the absorbent body 24. This allows for a high degree of air exchange with minimal dampness on the garment facing surface of the outer cover 20.

In the illustrated figures, the ventilation layer 32 is arranged in a direct, contacting liquid communication with the absorbent body 24. The ventilation layer 32 can be operably connected to the outer cover 20 with a pattern of adhesive, such as a swirl adhesive pattern. In addition, the ventilation layer 32 can be operably connected to the absorbent body 24 with a pattern of adhesive. The amount of adhesive add-on should be sufficient to provide the desired levels of bonding, but should be low enough to avoid excessively restricting the movement of air and vapor from the absorbent body 24 and through the outer cover 20.

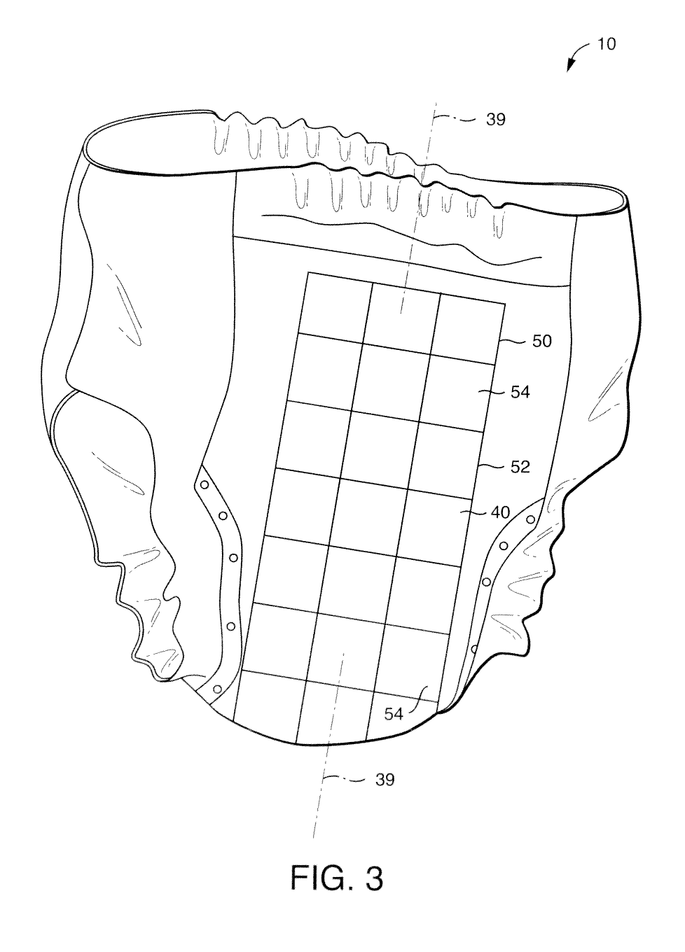

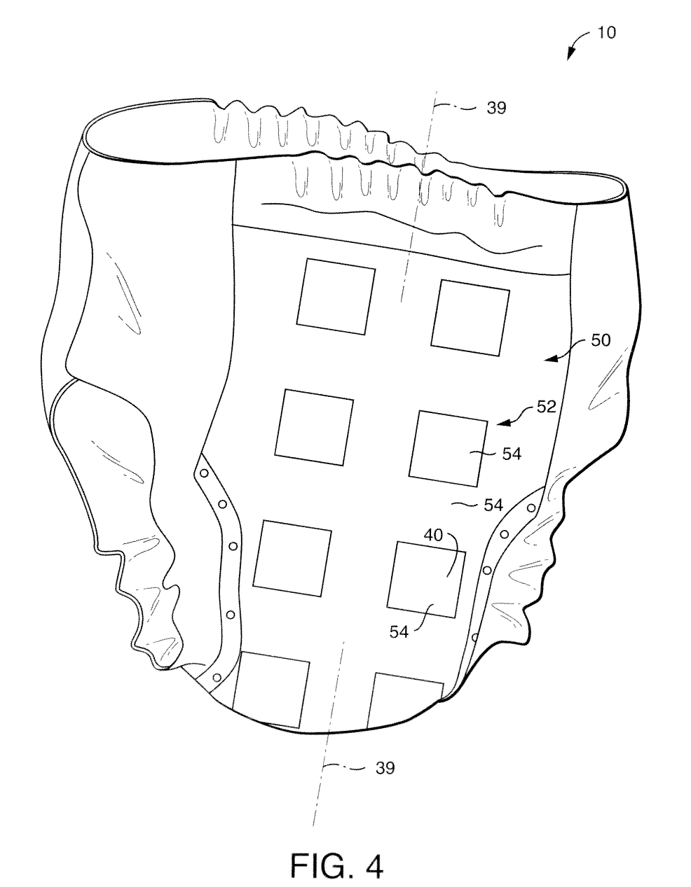

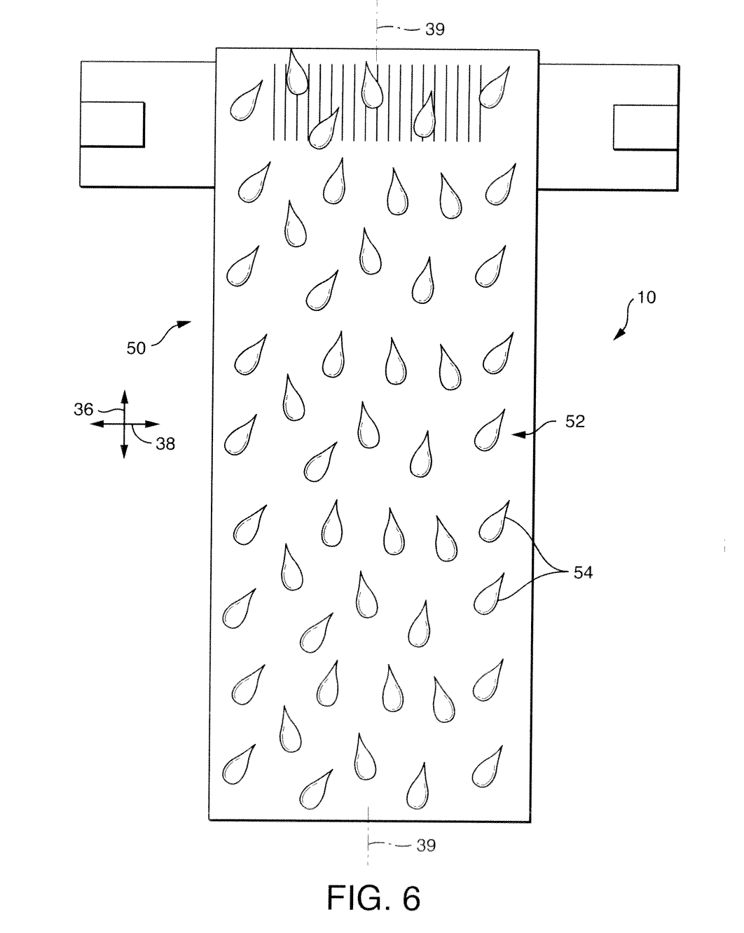

As illustrated in FIGS. 3-10, a disposable absorbent article 10 of the present disclosure includes a longitudinal centerline 39 and a urine insult volume indicator 50. The urine insult volume indicator 50 is a two-dimensional pattern 52 having a plurality of segments 54. In various aspects, the pattern 52 can be a checkerboard, a table with rows and columns, rows of various objects or characters, or any suitable pattern, including those illustrated in FIGS. 3 and 4. In these aspects, each checkerboard square, table cell, object, or character is a segment 54 of the pattern 52. Segments 54 can be outlined or framed such as FIGS. 3 and 4. In other aspects, the segments 54 can be without a frame or outline and appear adjacent to or in the same pattern 52 as outlines or frames, as shown in FIG. 4. In other words, the segments 54 can be the spaces between frames, outlines, or objects. In one exemplary illustration in FIG. 6, the segments 54 can be either the water droplet images themselves, or the segments 54 can be the spaces between the water droplet images, with the water droplet images disposed as points of reference to calibrate the wetted area and the total area of the pattern 52. Segments 54 can also be frameless regions of the pattern 52.

The pattern 52 has a longitudinal extent, which is the linear length of the pattern 52 in the longitudinal direction 36, and a transverse extent, which is the linear width of the pattern 52 in the transverse direction 38. One or more segments 54 of the pattern 52 can be disposed to intersect the longitudinal centerline 39, and one or more segments 54 can be disposed in a spaced apart relation from the longitudinal centerline 39. The area of a pattern 52 is generally taken to be the smallest area encircling all of the segments 54. The pattern 52 does not need to be centered in either the longitudinal or transverse directions 36, 38; the pattern 52 can also be shifted in any direction, particularly in the longitudinal direction 36, to accommodate gender differences in different article designs.

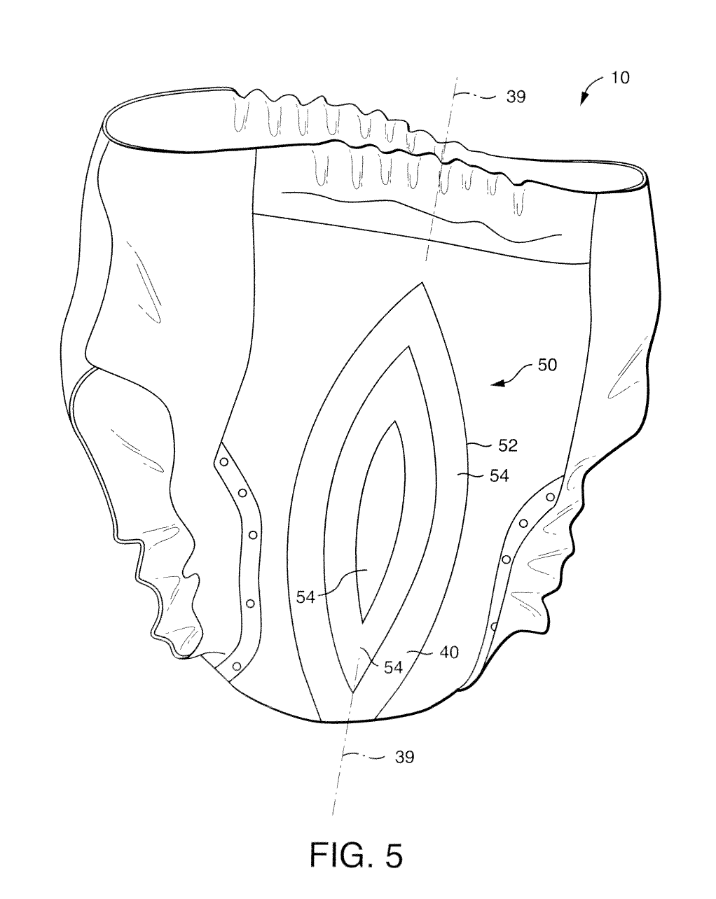

In other aspects of the present disclosure, the pattern 52 can be concentric figures such as circles, ovals, or ovoid figures. In these aspects, the longitudinal extent is the linear diameter or length of the pattern 52 in the longitudinal direction 36, and the transverse extent is the linear diameter or width of the pattern 52 in the transverse direction 38. An example of concentric pattern 52 is schematically illustrated in FIG. 5.

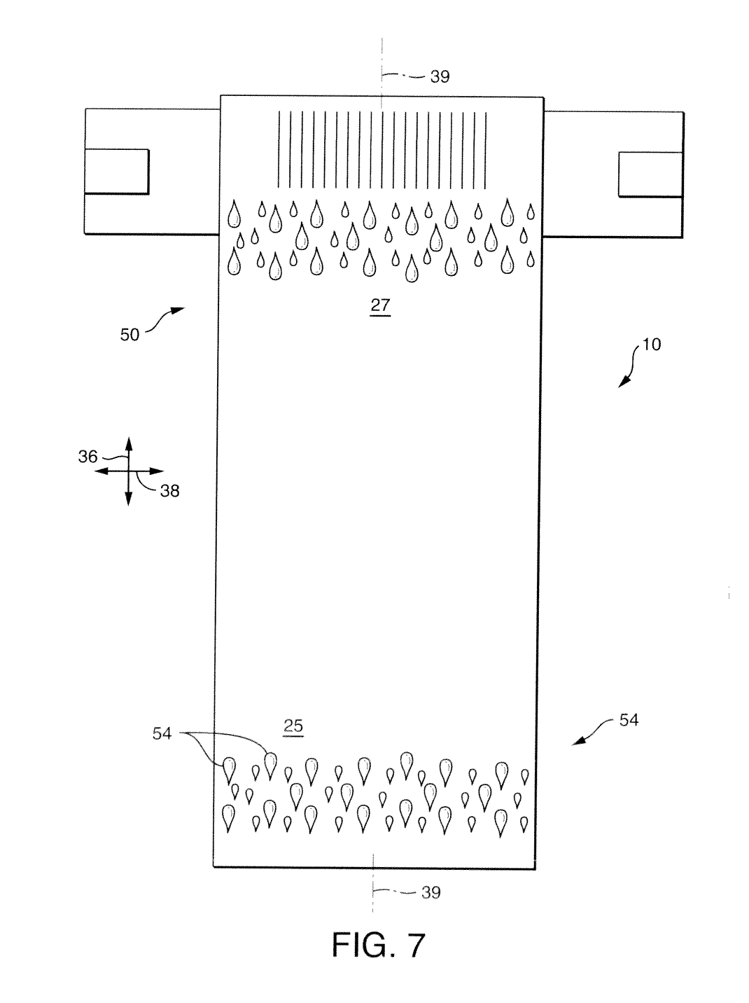

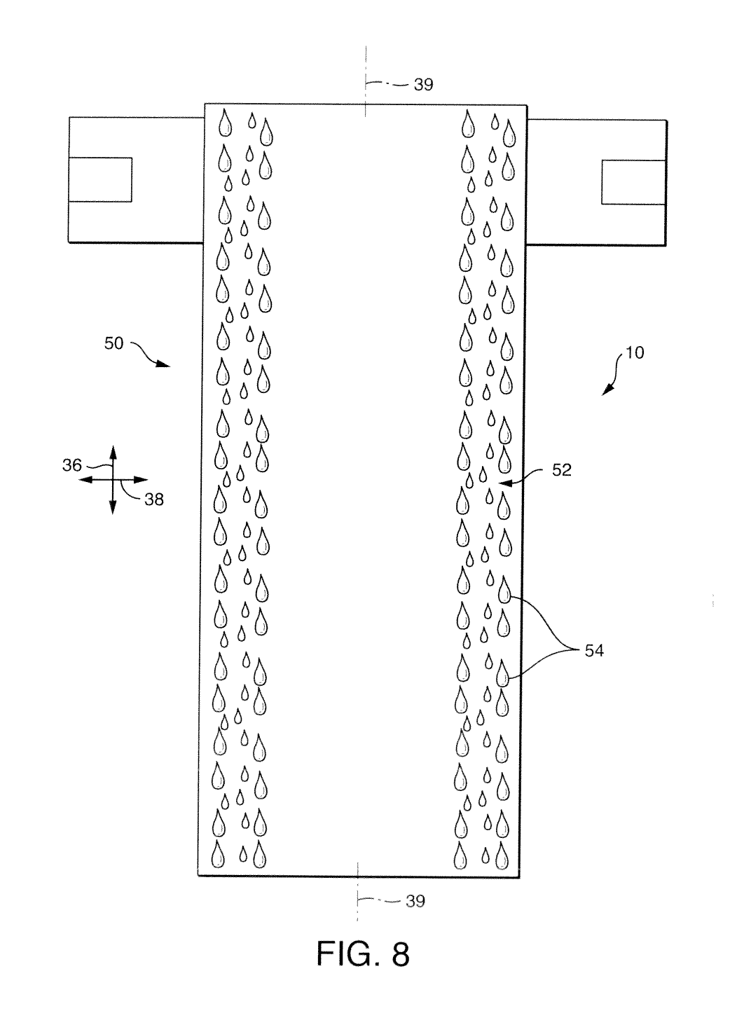

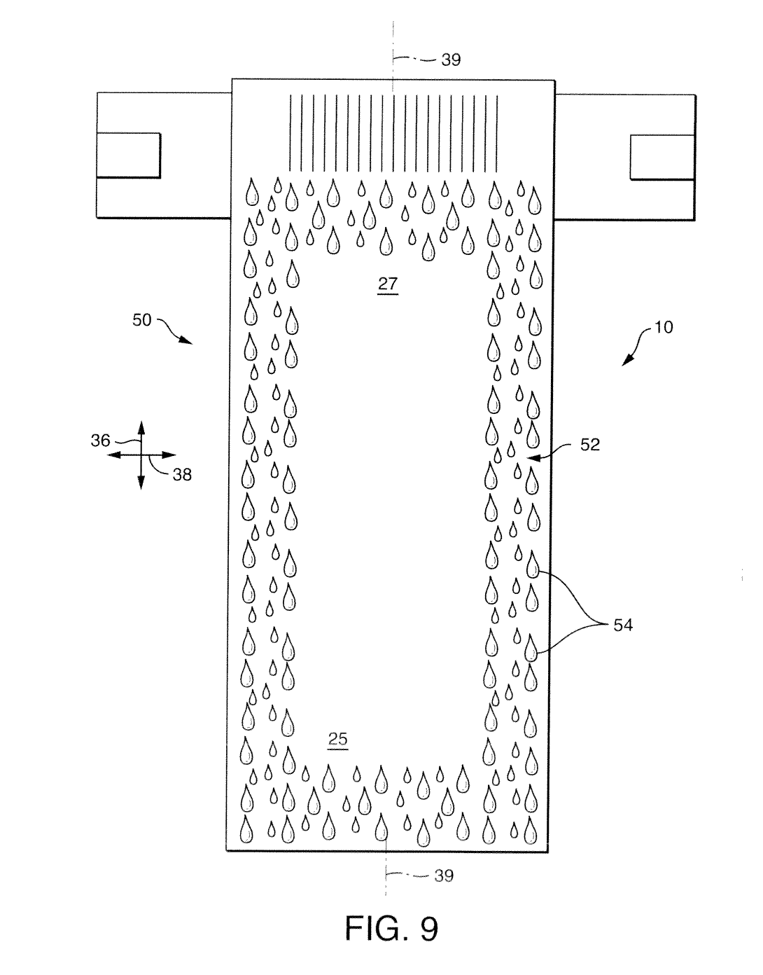

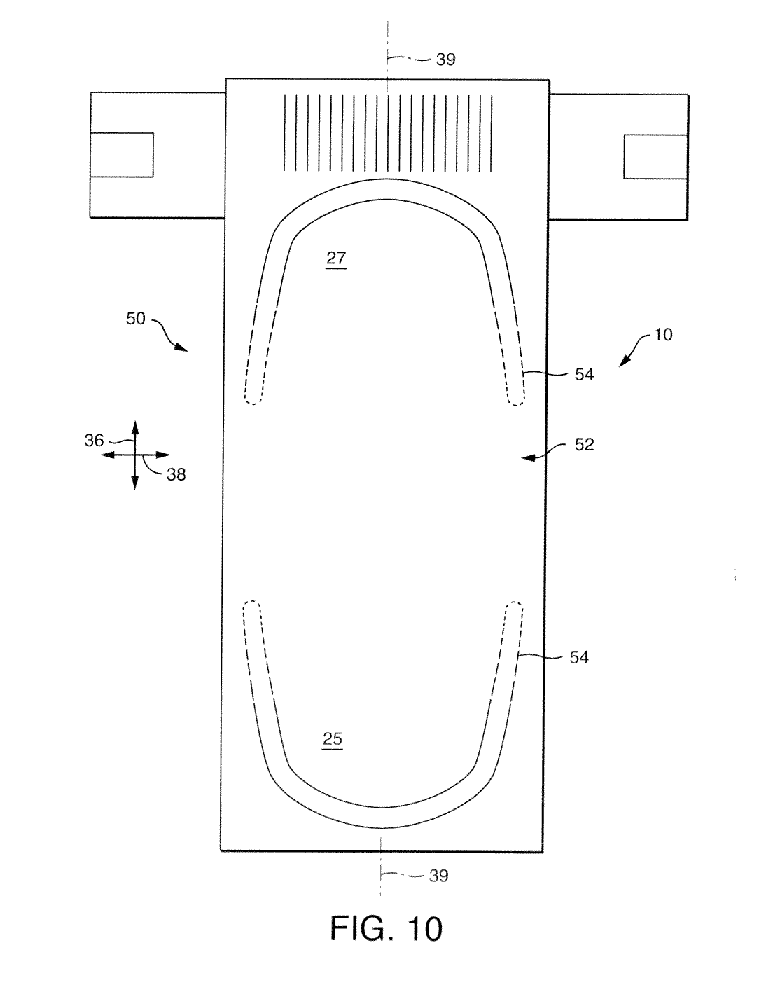

In other aspects of the present disclosure, the pattern 52 can include depictions of objects, characters, geometric shapes, or any other suitable graphical elements (each being a segment 54) arranged in a repeating rows and/or columns, or arranged apparently or actually randomly. For example, FIG. 6 shows a pattern 52 of water droplet images arranged apparently randomly. In alternate aspects of the present disclosure, a pattern 52 including depictions of objects, characters, geometric shapes, or any other suitable graphical elements (each being a segment 54) can be disposed toward the front waist region 25, the back waist region 27, the side or end margins of the article 10, or any combination of these. For example, FIG. 7 illustrates a pattern 52 of water droplet images arranged in the front and back waist regions 25, 27. FIG. 8 illustrates a pattern 52 of water droplet images arranged along the side margins of the article 10. FIG. 9 illustrates a pattern 52 of water droplet images arranged in the front and back waist regions 25, 27 and along the side margins of the article 10. FIG. 10 illustrates a pattern 52 with horseshoe-shaped segments 54 in each of the front and back waist regions 25, 27.

In still other aspects of the present disclosure, the pattern 52 can include graphical and/or textual elements that replace or supplement other segments 54. The outer cover 20 or other structure can be printed with graphical and/or textual elements that supplement the pattern 52.

In alternate aspects of the present disclosure, the pattern 52 can be an ordered or random display of graphical and/or textual elements that change appearance upon being wetted. In these aspects, each graphical and/or textual element can be considered a segment. In one aspect, the graphical and/or textual elements cover a portion of the article 10. In another aspect, the display of graphical and/or textual elements generally coincides with the area of the absorbent body 24. In still another aspect, the display of graphical and/or textual elements coincides only with the longitudinal ends and/or transverse sides of the absorbent body 24.

The pattern 52 is printed or otherwise disposed between the absorbent core 24 and the outer surface 23 such that the pattern 52 is visible from the outer surface 23 of the article 10 either before being wetted, after being wetted, or both. The pattern 52 is in fluid contact with the absorbent core 24 either directly or through an intermediate material or intermediate materials. Each segment 54 includes a colorant 40 as described herein. The colorant 40 and therefore a segment 54 changes appearance when wetted. The colorant 40 can change from one color to another, from one color to no color, or from no color to a color. Segments 54 need not be identical in their colorants 40 or in the manner each segment 54 changes appearance.

In one aspect of the present disclosure, the pattern 52 is disposed on the outer cover 20, particularly on an absorbent-facing side of the outer cover 20. In other aspects, the pattern 52 can be disposed on the absorbent core 24, on the ventilation layer 32, on a wrapsheet, on the surge management layer 34, or on an additional layer of material disposed between the outer cover 20 and the absorbent core 24.

In another aspect of the present disclosure, the absorbent body 24 or any other structure between the bodyside liner 22 and the outer cover 20 can include apertures or pores therethrough to allow an insult to more quickly reach the garment-facing side of the absorbent body 24. In this manner, the insult can contact the insult volume indicator 50 more quickly, allowing the insult volume indicator 50 to indicate and the caregiver to observe the occurrence of even very small insults.

The absorbent core 24 includes a longitudinal end in the front waist region 25 and a longitudinal end in the back waist region 27. The absorbent core 24 also includes transverse sides. In one aspect of the present disclosure, the longitudinal extent of the pattern 52 generally coincides with the longitudinal ends of the absorbent core 24, and the transverse extent of the pattern 52 generally coincides with the transverse sides of the absorbent core 24.

In various aspects, the colorant 40 can be employed to estimate a volume of an insult or a void. Particularly if the article 10 includes a surge management layer 34 that is designed to spread the insult laterally and longitudinally, the spread of the insult becomes somewhat uniform and the volume can be estimated by using the appearance change of the pattern 52. The pattern 52 is selected and arranged such that segments 54 change appearance primarily in the longitudinal direction 36 as the article 10 is wetted. Segments 54 can also be selected and arranged such that segments 54 change appearance in the transverse direction 38 as the article 10 is wetted. The volume of liquid insult in the article 10 can be correlated to the number of segments 54 that change appearance, the fraction/percentage of the area of the pattern 52 that has changed appearance, the fraction/percentage of the longitudinal extent of the pattern 52 that has changed appearance, or the fraction/percentage of the transverse extent of the pattern 52 that has changed appearance when wetted.

For example, in general, a change in appearance of half of the segments 54 indicates that half the insult-absorbing capacity of the article 10 has been reached. In another example, for a pattern 52 that includes segments 54 arranged in rows, in general the number of rows in the longitudinal direction 36 that have changed appearance as a fraction of the total number of rows in the longitudinal extent indicates the fraction of insult-absorbing capacity of the article 10 that has been reached. To simplify this example, studies described below have shown that the first row that includes a segment 54 that has changed appearance is largely consistent, meaning that only the last row that includes a segment 54 that has changed appearance need be used to determine the extent of loading. In still another example, for a pattern 52 that includes segments 54 arranged in columns, in general the number of columns in the transverse direction 38 that have changed appearance as a fraction of the total number of columns in the transverse extent indicates the fraction of insult-absorbing capacity of the article 10 that has been reached.

For an article 10 of a given design, the fraction of insult-absorbing capacity used as indicated by the change in appearance of the pattern 52 can be directly correlated to a specific urine insult volume, as described further in the example below.

In other aspects of the present disclosure, the graphical and/or textual elements described above can be used to indicate to a caregiver a specific insult volume. For example, the segments 54 of the pattern 52 can be a scale of zero to 100 cc printed along the longitudinal centerline, where the printing includes the colorant 40. As the article 10 is insulted, the graphical and/or textual elements change appearance, immediately indicating to a caregiver the volume of fluid insult. In another example, the segments 54 of the pattern 52 can be a scale of zero to 100 percent printed along the longitudinal centerline 39, where the printing includes the colorant 40. It should be noted that the zero point in such a scale is not necessarily positioned in the crotch region 29 of the article 10. Wearers and therefore articles 10 of different sizes, and wearers of different genders, tend to have different target zones of initial insult contact with the absorbent core 24. The scales in these examples can be calibrated and positioned such that, for example, an indicated 50 percent absorbent capacity insult actually is indicated as 50 percent on the pattern 52. Again, studies described below have shown that the first row that includes a segment 54 that has changed appearance is largely consistent, meaning that only the last row that includes a segment 54 that has changed appearance need be used to determine the extent of loading.

In alternate aspects of the present disclosure, the scales described above can be printed with generally insoluble ink in a manner that complements segments 54 that change appearance when wetted. In a specific example, the scale of 0 to 100 percent can be printed on a surface of the outer cover 20, with segments 54 including the colorant 40 printed on the same or another surface of the outer cover 20 such that the pattern 52 appears to overlie the scale, or vice versa, allowing a caregiver to see the segments 54 that have changed appearance in conjunction with a generally permanent scale.

The colorant 40 used in or as each segment 54 can include any material that can indicate the desired property when an insult is received. The colorant can include a dye, such as bromocresol green; m-cresol purple; cresol red; chlorophenol red; bromothymol blue; bromopyrogallol red; bromoxylenol blue; methylene blue; bromophenol blue; monoazo dyes (such as acid alizarin violet N); monoazo pyrazoline dyes (such as acid yellow 34); diazo dyes (such as acid black 24); anthraquinone dyes (such as acid black 48); amphoteric anthraquinone dyes (such as acid blue 45); triphenylmethane dyes (such as acid fuchsin); phthalein type dyes (such as o-cresolphthalein); xanthene dyes (such as 2′,7′-dichlorofluorescein eosin B); heterocyclic acridine aromatics (such as acridine orange); diphenylmethane dyes (such as auramine O); triphenylmethane dyes (such as basic fuchsin); cationic thiazine dyes (such as azure C); cationic anthraquinone dyes (such as basic blue 47); phthalocyanine type dyes (such as copper phthalocyanine); quaternized phthalocyanine type dyes (such as alcec blue); cationic polymethine dyes (such as astrazon orange G); anthraquinone type (such as alizarin); the neutral complex dyes (such as azure A eosinate); the terpene type dyes (such as trans-beta-carotene); and so forth, as well as combinations including at least one of the foregoing dyes. The colorant 40 can also include leuco dyes and related chemistries such as those described in U.S. Patent Application Publication Nos. 2010/0114047 A1 and 2010/0030173 A1, and colorant chemistries such as those described in U.S. patent application Ser. No. 12/825,877, which are incorporated herein by reference to the extent they do not conflict herewith. The colorant 40 can also include colorant chemistries and functionalities such as those described in U.S. patent application Ser. Nos. 61/419,981; 12/491,701; 12/637,217; 12/640,604; 12/503,380; 12/503,398; and 12/503,364; the contents of which are incorporated herein by reference to the extent that they are consistent (i.e., not in conflict) herewith. Desirably, the colorant(s) 40 are U.S. FDA approved.

The colorant 40 can be encapsulated in a material that is soluble in urine. Suitable urine or water soluble materials include, but are not limited to, cellulose-based polymeric materials (such as ethyl cellulose); carbohydrate-based materials (such as starches, sugars, and materials derived therefrom, e.g., dextrins and cyclodextrins); and so forth, as well as combinations including at least one of the foregoing materials. When the colorant 40 is contacted by urine, the encapsulating material dissolves releasing the dye contained therein. The visual presence of the dye indicates that urine is present. The amount of urine necessary to create a visual indication is at least about 1 to 15 cc, 1 to 10 cc, 1 to 5 cc, or at least 1 cc.

The colorant 40 can alternatively or additionally include one or more materials that can indicate the presence of a substance in or condition of the urine. For example, pH levels in the urine can be indicated by using methyl red, bromthymol blue, and phenolphthalein. The pH range 5 to 9 yields a color gradation from orange to yellow to green to blue. Any combination of the materials described herein can be encapsulated in a water or urine soluble encapsulant. More than one colorant can be included in any single absorbent article 10.

The disclosure is further illustrated by the following non-limiting example.

Two studies, including a mannequin study, were completed to develop a fullness indicator for articles 10, in this case for U.S. size 3 articles. The articles 10 were hand modified with an outer cover 20 printed with an ink including a colorant 40 in a checkerboard pattern (9×36 squares). The checkerboard pattern was designed to cover the entire area of the absorbent core 24 in the article 10 to assess fluid distribution. The proprietary test ink was a color-changing wetness indicator based on pH indicator technology. The ink changes color from yellow to blue when wet. The type of ink used is largely immaterial to the results as long as the ink includes a colorant 40 of the types described herein. The modified articles 10 were placed on mannequins in one study and subjected to various insult amounts.

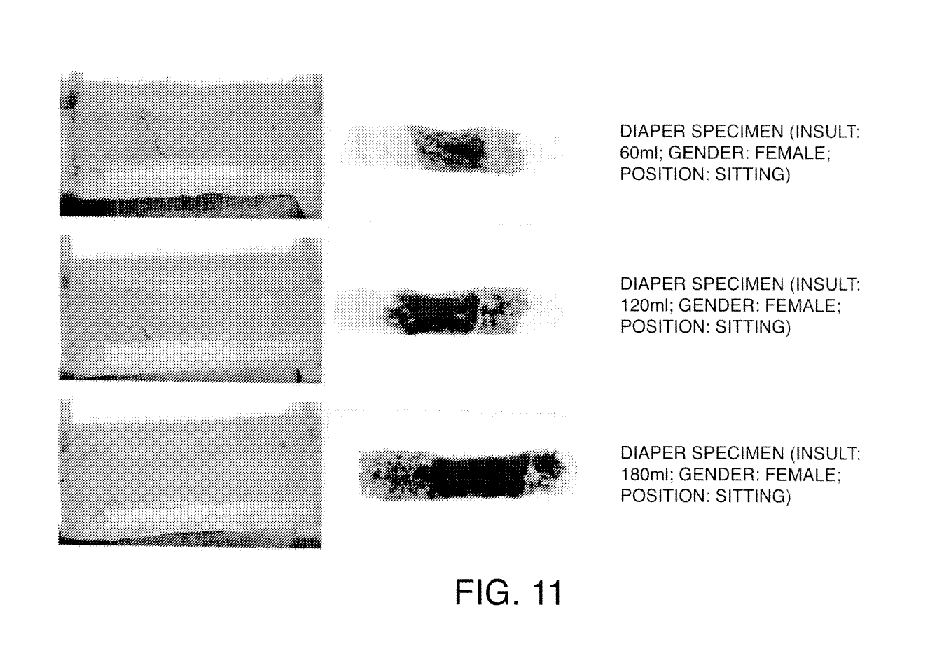

Results from the mannequin study are illustrated in FIG. 11. For each of three samples, an article 10 was modified with a checkerboard pattern of square segments. Each insulted article 10 was then photographed and x-rayed, with the insulted area in each outlined in black marker. A comparison of the photograph and x-ray for each sample shows that wetted length and wetted area data obtained from photographed checkerboard outer covers 20 matched well with wetted length and wetted area data from the corresponding x-ray images of the same articles 10. See FIG. 11. Therefore, the checkerboard pattern of this example using proprietary ink tracked a wetted area well. As can be seen in FIG. 6, there was a strong correlation between the number activated checkerboard squares (or area) and loading level.

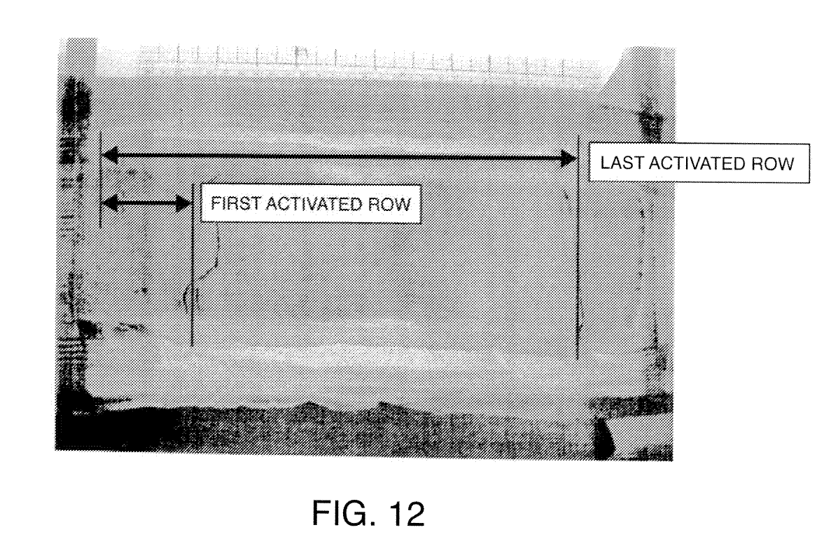

This correlation was further confirmed as illustrated in FIGS. 12-16. As shown in FIG. 12, sample articles 10 treated under the same conditions described above were photographed with the wetted area outlined with black marker. The wetted area in each case was determined by counting the number of squares that changed appearance. The wetted length in each case was determined by counting the number of wetted rows from the first row that included a square that changed appearance to the last row that included a square that changed appearance.

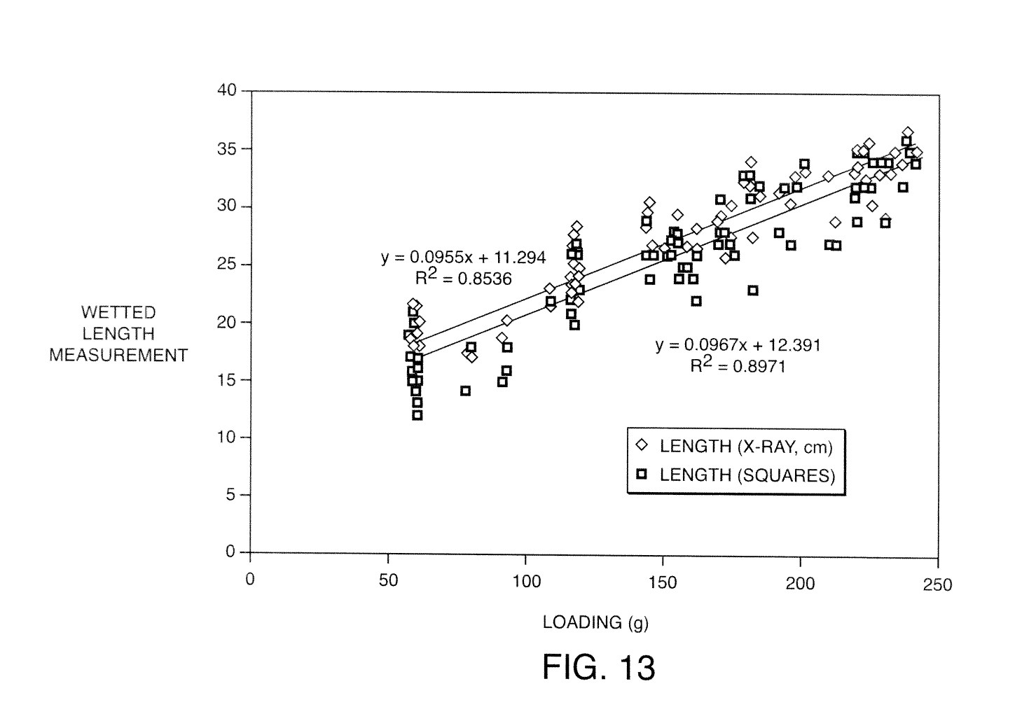

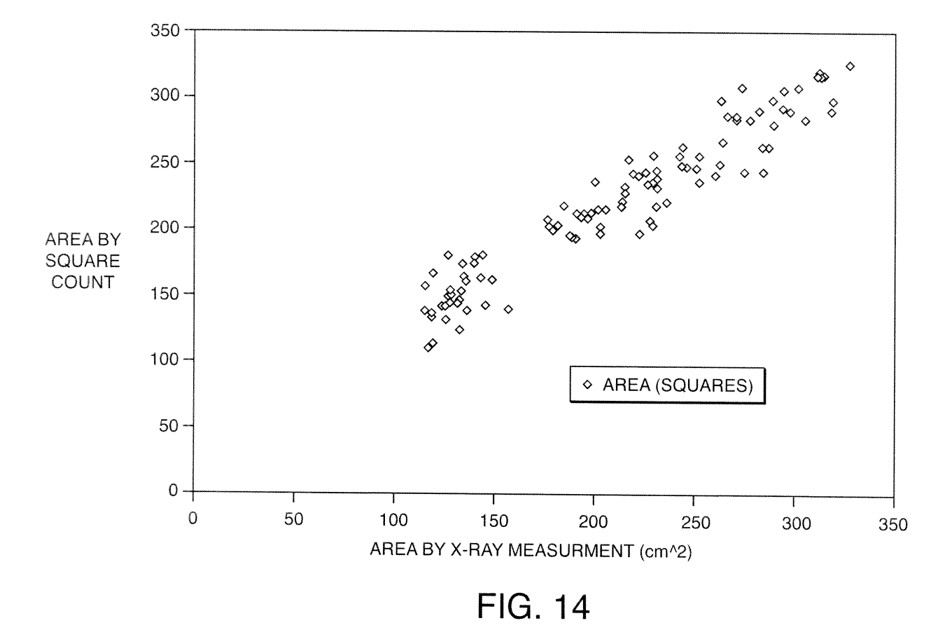

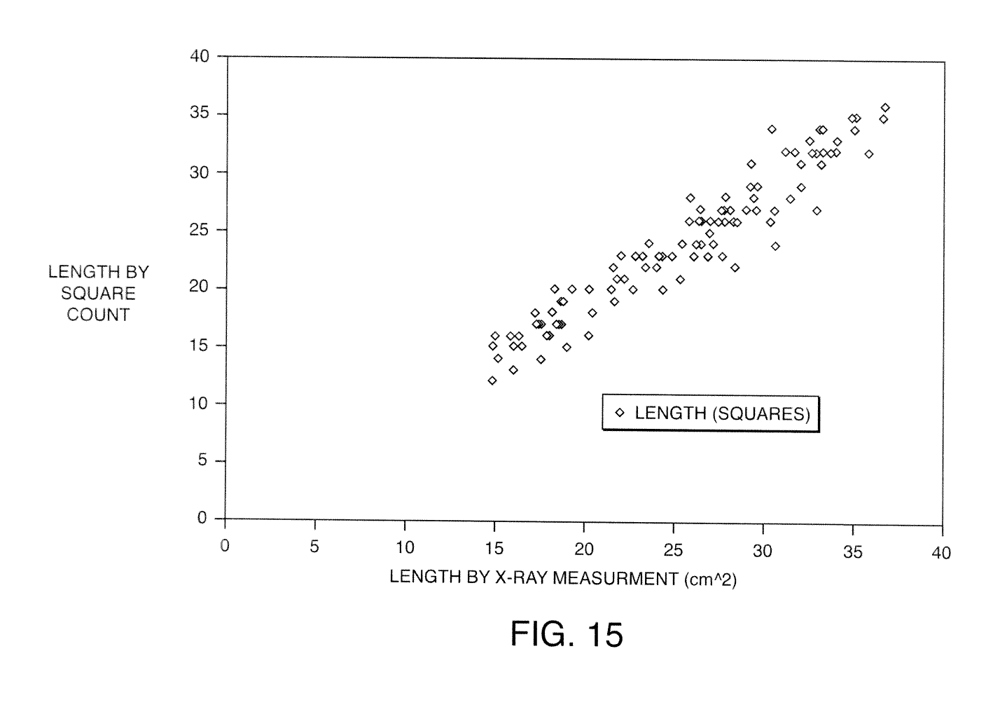

Data collected are summarized in FIGS. 13-16. FIG. 13 illustrates the linear correlation between wetted length and insult amount as obtained from x-ray analysis and from counting the number of squares that changed appearance. FIG. 14 illustrates the linear correlation between wetted area as obtained from x-ray analysis and wetted area as obtained from counting the number of squares that changed appearance. FIG. 15 illustrates the linear correlation between wetted length as obtained from x-ray analysis and wetted length as obtained from counting the number of rows with squares that changed appearance (activated rows).

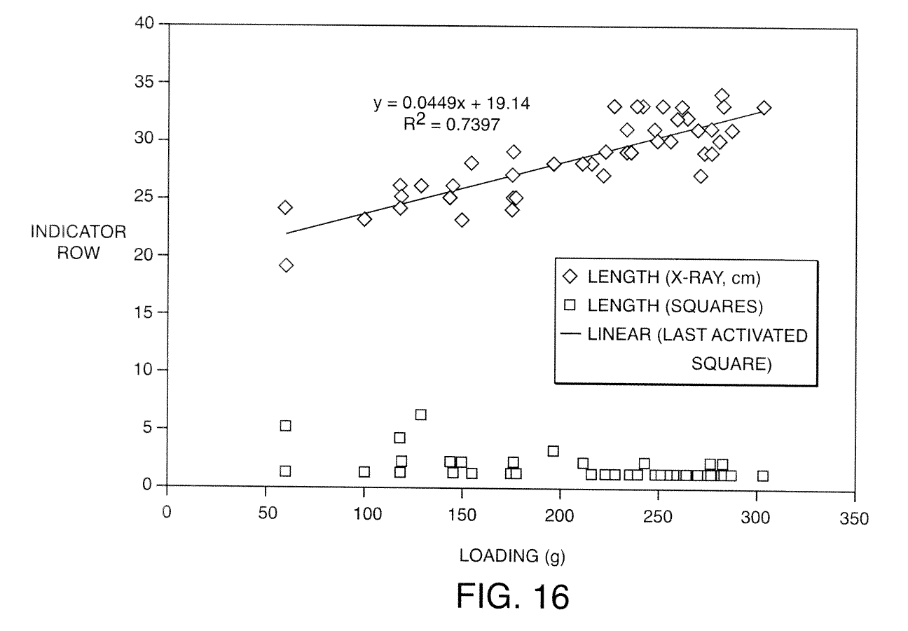

FIG. 16 illustrates the linear correlation between insult amount and the first and last rows with squares that changed appearance (first and last activated rows/squares). This means that as loading increased, wetted length increased and it was captured by the last activated row (in that the first activated row remained relatively constant). As a result, the last activated row can be used to determine how much urine insult volume has been introduced in an article 10. In general, determining how much urine insult volume has been introduced can mean that the urine insult volume is calculated or estimated, based on the type of pattern and other printing used, and on the sophistication of the one doing the determining.

Infants, e.g., greater than or equal to about 6 months old, produce insults of around 60 cc. However, newborns tend to have much smaller insults of around 10 to 15 cc, or even less. The absorbent articles 10 disclosed herein can be used to identify the existence of a very small volume of an insult (e.g., less than or equal to about 10 cc, or, more specifically, less than or equal to about 5 cc, and even more specifically about 1 cc). The absorbent articles 10 described herein can detect “off target” insults as well, meaning insults that do not directly impact the target zone described above. Finally, the absorbent articles 10 disclosed herein can detect the presence of various substances that can be found in urine.

When introducing elements of the present disclosure or the preferred aspect(s) thereof, the articles “a,” “an,” and “the” are intended to mean that there are one or more of the elements. The terms “comprising,” “including,” and “having” are intended to be inclusive and mean that there can be additional elements other than the listed elements.

The disclosure has been described with reference to various specific and illustrative aspects and techniques. However, it should be understood that many variations and modifications can be made while remaining within the spirit and scope of the disclosure. Many alternatives, modifications and variations will be apparent to those skilled in the art in light of the foregoing description. Accordingly, this disclosure is intended to embrace all such alternatives, modifications, and variations that fall within the spirit and scope of the appended claims.

We claim:

1. A disposable absorbent article having a urine insult volume indicator and a longitudinal centerline, the article comprising:
a liquid permeable inner surface for facing a wearer;
an outer cover having an outer surface for facing away from the wearer;
an absorbent body disposed therebetween;
a front waist region, a back waist region, and a crotch region extending longitudinally between and interconnecting the front and back waist regions; and
a two-dimensional pattern having a plurality of segments, a longitudinal extent, and a transverse extent, the pattern disposed between the absorbent core and the outer surface, wherein the pattern is in fluid contact with the absorbent core, and wherein a segment changes appearance when wetted and is visible from the outer surface when wetted.

2. The article of claim 1, wherein the pattern is disposed on the outer cover.

3. The article of claim 1, wherein the pattern is disposed on the absorbent core.

4. The article of claim 1, further comprising a layer of material disposed between the absorbent core and the outer cover, wherein the pattern is disposed on the layer of material.

5. The article of claim 1, the absorbent core further comprising a front waist longitudinal end and a back waist longitudinal end, wherein the pattern extends from the front waist longitudinal end to the back waist longitudinal end.

6. The article of claim 1, the absorbent core further comprising first and second transverse sides, wherein the pattern extends from the first transverse side to the second transverse side.

7. The article of claim 1, the absorbent core further comprising a front waist longitudinal end and a back waist longitudinal end, wherein the pattern is disposed only adjacent the front waist longitudinal end and the back waist longitudinal end.

8. The article of claim 1, the absorbent core further comprising first and second transverse sides, wherein the pattern is disposed only adjacent the first and second transverse sides.

9. The article of claim 1, the absorbent core further comprising a front waist longitudinal end and a back waist longitudinal end and first and second transverse sides, wherein the pattern is disposed only adjacent the front waist longitudinal end and the back waist longitudinal end and the first and second transverse sides.

10. The article of claim 1, wherein at least one segment is disposed to intersect the longitudinal centerline and at least one segment is disposed in a spaced apart relation from the longitudinal centerline.

11. The article of claim 1, wherein the pattern is disposed such that the pattern is biased toward the front waist region.

12. The article of claim 1, wherein the pattern includes concentric segments.

13. The article of claim 1, wherein the pattern includes a checkerboard pattern.

14. The article of claim 1, wherein the pattern includes a random arrangement of segments.

15. The articles of claim 1, wherein the segments are frameless.

16. A method for providing a disposable absorbent article having a urine insult volume indicator and a longitudinal centerline, the method comprising:
manufacturing a disposable absorbent article including a liquid permeable inner surface for facing a wearer; an outer cover having an outer surface for facing away from the wearer; an absorbent body disposed therebetween; and a two-dimensional pattern having a plurality of segments, a longitudinal extent, and a transverse extent, the pattern disposed between the absorbent core and the outer surface, wherein the pattern is in fluid contact with the absorbent core, and wherein a segment changes appearance when wetted and is visible from the outer surface when wetted; and
correlating the number of segments that change appearance upon wetting to a urine insult volume resident in the absorbent core.

17. The method of claim 16, wherein the percent of fullness of the absorbent core is determined from the ratio of the number of segments along the longitudinal extent that have changed appearance to the total number of segments along the longitudinal extent.

18. The method of claim 16, wherein the percent of fullness of the absorbent core is determined from the ratio of the number of segments that have changed appearance to the total number of segments in the pattern.

19. The method of claim 16, wherein the percent of fullness of the absorbent core is determined from the ratio of the number of segments along the transverse extent that have changed appearance to the total number of segments along the transverse extent.

20. The method of claim 16, wherein the pattern has a total area, wherein a wetted portion of the pattern has a wetted area, and wherein the percent of fullness of the absorbent core is determined from the ratio of the wetted area to the total area.

21. The method of claim 20, wherein the wetted area is determined by counting the number of segments that have changed appearance.

22. The method of claim 16, wherein the article includes a front waist region and a back waist region, and wherein the pattern is disposed such that the pattern is biased toward the front waist region.
